(19) United States
(12) Patent Application Publication
(10) Pub. No.: US 2026/0355483 A1
(43) Pub. Date: Nov. 12, 2026

(54) Dermal Media Application System

(71) Applicants: Alexandra Rose Martello, Chicago, IL (US); Matthew T. Cruz, Chicago, IL (US)

(72) Inventors: Alexandra Rose Martello, Chicago, IL (US); Matthew T. Cruz, Chicago, IL (US)

(21) Appl. No.: 19/390,804

(22) Filed: Nov. 17, 2025

Related U.S. Application Data
(63) Continuation of application No. 19/203,447, filed on May 9, 2025.
Application No. 19/203,447 is a continuation of application No. PCT/US25/28556, filed on May 9, 2025.

(51) Int. Cl. A61M 37/00

(52) U.S. Cl. CPC A61M 37/0015; A61M 2037/0023; A61M 2205/332; A61M 2205/3331

(57) ABSTRACT

The present system is related to a dermal device for applying therapy to the dermis of a patient. The device includes a main body that houses a controller and one or more actuators. A needle cartridge and media cartridge are separate and connectable to the main body. The media cartridge and needle cartridge interface with the main body such that the controller of the main body drives the one or more actuators to induce pressure to the media cartridge and flow a media from the media cartridge to the needle cartridge while also reciprocating needles that apply the media to a target site on the dermis of a patient.

### INCORPORATION BY REFERENCE

This application claims priority to and incorporates by reference in its entirety and for all purposes U.S. patent application Ser. No. 19/203,447 filed on behalf of Alexandra Rose Martello and Mark T. Cruz in the United States Patent Office on May 9, 2025 which claims priority to and incorporates by reference in its entirety and for all purposes PCT patent application serial number PCT/US25/28556 filed on behalf of Alexandra Rose Martello and Mark T. Cruz in the United States Patent Office on May 9, 2025.

### BACKGROUND

Intradermal delivery of media is commonly used in medical and cosmetic therapies. Dermal devices may be used to penetrate the top layer of skin to provide an avenue for delivery of media to the body without penetrating into the muscle. Microneedling devices are one type of dermal devices that are used for a minimally invasive procedure which treats a variety of conditions including but not limited to: acne scars, wrinkles, fine lines, stretch marks, hyperpigmentation, enlarged pores, cellulite and hair loss. Microneedling procedures are performed using dermal microneedling devices. Other uses of dermal devices include skin rejuvenation, body contouring, skin tightening, lymphatic drainage and permanent make up procedures. The devices operate by utilizing a needle that pierces partially into the skin. Unlike a hypodermic needle used for injecting medication, such as insulin or a vaccination, which pierces completely through the skin and into veins or muscle, dermal devices are restricted in the depth to which the needles pierce so as to remain focused at the skin level.

Another dermal therapy device is a mesotherapy device. A mesotherapy device punctures needles into the target skin area using a point-by-point technique. Usually, mesotherapy is conducted at a slower needle puncture rate than microneedling therapies.

Permanent makeup therapies are also performed using a dermal device. During a permanent makeup procedure, the dermal device needle tip is dipped into a liquid before administering the needle into the target skin area. Continuously dipping the permanent makeup dermal device tip into ink during the procedure causes the procedure to be performed at a slower rate and leads to excessive pooling of ink on the surface of the skin.

It is highly desirable to inject media with more depth precision to the puncture site than a classic single use syringe. Current microneedling devices lack the control necessary to precisely administer the desired medium in a reliable, controlled manner. For example, a permanent makeup dermal device uses a reservoir of ink to dip into and capillary action associated with piercing skin with an ink-covered solid needle. A vacuum generated when the needle withdraws will draw the ink into the skin where it remains. In the process, excess ink pools on the skin's surface and must be perpetually wiped away and is wasted. Unlike other handheld dermal devices, the present invention is able to conduct a variety of treatments with precise control and provide the ability to quickly interchange needle and media cartridge types. Currently, the majority of dermal devices conduct one specific type of dermal therapy, and an individual skilled in the art will appreciate the present system's ability to conduct an array of therapies by having the ability to swap out media cartridges and needle cartridges.

### SUMMARY

The present device is a dermal media application system that is primarily used in dermal therapy procedures. It includes an array of reciprocating fine needles that puncture the skin to a preferred depth of 3 mm or less from the skin's surface. The puncturing of the needles into the skin instigates a healthy immune response (in the case of microneedling) and allows for the precise delivery of a desired media (for example a fluid state product such as a drug, collagen, permanent makeup, etc.). During the puncture, the array of needles introduces the media into the puncture site. Usually, the puncturing is performed 40-100 times per second. The present dermal system is designed to deliver media intradermally.

The present system is constructed in a unique, all-in-one, hand-held device. It does not require separate ink wells for dipping, external tubing to separate reservoirs of media, outside tubing, or hardwiring. Rather, all subassemblies required for appropriate operation are contained in a sleek handheld device with hot-swapable components that allow a user to quickly and efficiently update the media being supplied and work without having external systems (like tubing and wiring) interfere with the operation of the device or contact the patient receiving treatment by the device.

The system includes an array of hollow needles through which a media may flow. The needle array is arranged at the tip of the device, which generally has the shape of a cylinder, like a large pen. The hollow needles are connected to a flow circuit (which may be one or more tubes or channels through which fluid may flow). The flow circuit terminates at one or more ports that correspond with the port(s) of a media cartridge. A pressure system, which may be variable, induces pressure into the flow circuit to move the media from the cartridge through the flow circuit and through the needles. For example, a pump may be used to induce pressure into the cartridge, or to draw fluid from the cartridge, and into the flow circuit and then push the fluid from the flow circuit through the needles.

The system includes an actuator that operates to move the needles. For example, a motor may be used to reciprocate the needles. The actuator and pressure system work in conjunction with each other to reciprocate the needles and flow fluid when the needles are in the appropriate position. For example, when the system is being used, the needles are positioned proximate to a patient's skin, the actuator is activated to cause the needles to move away from the center of the system, toward the patient, and pierce into the patient's skin. As the needles penetrate, the fluid is flowed through the needles, out of the tips of the needles, and into the patient's skin. The needles are then retracted toward the center of the system and the flow of fluid stops. The process is then repeated. In an alternate embodiment, a media well surrounds one or more needles and, through surface tension of the media, holds the media in an orifice through which the needle penetrates. The orifice is supplied with media from the media cartridge and, as the needle passes through the orifice, it is coated with the media. Due to the surface tension, however, regardless of the orientation of the device, the media is prevented from dripping from the orifice and needle(s). In this way, the needle may be continuously supplied with an appropriate amount of media while preventing dripping and excess pooling of media at the application site.

A controller is integrated with the body of the system to control the operation of the actuator and the pressure of the system and to maintain the appropriate timing. Additional control functions may also be carried out. As noted, the system includes a media cartridge. The media cartridge affixes to the tip of the body. It mates with the fluid circuit which allows the media to flow from the cartridge through the needles. The cartridge is affixed in a toolless manner, such as by snap-fit engagement so that it may be easily removed and replaced without undue interruption in a particular dermal therapy procedure. This also allows the system to be quickly purged of media and swapped with alternative media during a procedure.

Additional aspects of the present system will be apparent with reference to the drawings.

### BRIEF DESCRIPTION OF THE DRAWINGS

FIG. 1 is a depiction of an embodiment of the dermal system displaying the general relationships of the body, needle cartridge, and media cartridge components.

FIG. 2 is a cross-sectional view of an embodiment of the dermal system.

FIG. 3 is a three-dimensional perspective view of select components of an embodiment of a needle cartridge.

FIG. 4A is a three-dimensional perspective view of select internal components of a bellows pump that may be adapted to move media through the dermal system.

FIG. 4B is a three-dimensional perspective view of the external component of the bellows pump.

FIG. 5A is a three-dimensional perspective view of one side of an embodiment of a media cartridge.

FIG. 5B is a three-dimensional perspective view of an alternate side of the embodiment of a media cartridge depicted in FIG. 5A.

FIG. 5C is a cross-sectional view of the embodiment of an internal reservoir of a media cartridge.

FIG. 5D is a perspective view of an internal reservoir of a media reservoir.

FIG. 6A is a perspective view of an embodiment of a de-bubbler.

FIG. 6B is a cross-sectional view of an embodiment of a de-bubbler.

FIG. 7A is a cross-sectional view of an alternate embodiment of a de-bubbler.

FIG. 7B is a perspective view of the de-bubbler depicted in FIG. 7A.

FIG. 7C is a top view of the de-bubbler depicted in FIG. 7A.

FIG. 8 is a block diagram of select components of an embodiment of the dermal system.

FIG. 9 is a cross-sectional view of an embodiment of the needle manifold assembly.

FIG. 10 is a cross-sectional view along A-A of an embodiment of a media well utilized in conjunction with the embodiment of FIG. 9.

### DETAILED DESCRIPTION

Throughout the specification, wherever practicable, like structures will be identified by like reference numbers. In some figures, components, such as additional electrical connections, tubing, fasteners, or shrouds have been omitted for clarity in the drawings. Unless expressly stated otherwise, connections between components may be made directly or indirectly. Unless expressly stated otherwise, the term “or” means “either or both” such that “A or B” includes A alone, B alone, and both A and B together.

According to the present system, a handheld dermal media application system may perform one or more skin treatments on a person's skin. The term “skin treatments” is a broad term and is not limited to skin rejuvenation, body contouring, skin tightening, skin removal, skin abrasion (e.g. dermabrasion, microdermabrasion, etc.), ablating or slicing the skin, skin stimulation, and the like. The handheld dermal system can treat a target skin area while also applying a treatment media to the patient. The handheld dermal system may be used at a hospital, healthcare physicality, residences or any other suitable location. The user is typically an aesthetician (e.g., an aesthetician allowed to perform microdermabrasion), doctor, or other medical personnel, such as a physician assistant or nurse practitioner that perform a treatment on another party. In some cases, the user is the person whose skin is being treated. It should be understood that the present system is not limited to being used on humans, and may be used with animals. However, the discussion of the system may generally refer to the party being treated as a “person” for simplicity.

As used herein, the term “media” is a broad term and includes substances in a fluid state, such as but not limited to: medications, gels, liquids (e.g. serums, cleansers, acids, water, saline, toxins, ink, botanical extracts). In certain embodiments multiple media may be delivered into the target area of the skin.

The handheld dermal system allows for sterile application of media while performing skin treatments or therapies. The needle cartridges may be sterilized during manufacture and packaged in sterilized packaging. The housing portion of the system is reusable, and the sterilized needle cartridge may be affixed to the housing at the facility where the dermal procedure will be performed. The handheld dermal system is also configured to administer a controlled and repeatable volume of media from a media cartridge through a needle cartridge and into the target dermal area.

The handheld dermal system is comprised of three main components, a reusable housing that forms the basic structure of the device, a needle cartridge that connects to the housing, and a media cartridge that connects to the housing. In some embodiments, the needle cartridge and media cartridge are formed as a single component, but for ease of reference they will be discussed separately. When assembled, the dermal system is a fully self-contained apparatus that is intended for single hand use by an operator. The operator is able to select the particular type of needles for a treatment, the corresponding media within a media cartridge, connect both to the universal housing, and begin treatments.

The housing, which may be a plastic or metal tubular structure, forms the main body of the system. It houses an actuator, such as a rotary motor. It may also include a speed indicator, a needle speed adjustor, a needle height adjustor, a media flowrate adjustor, a media flowrate indicator, a force sensor, a force sensing display, a controller, and a power source, such as battery. The various adjustments may be made through communication with the controller which relays the control signals to the appropriate actuator. For example, a switch on the body may be selected to cause the controller to relay a signal to the motor to change speeds. A sensor adapted to sense pressure experienced by the needle cartridge tip will trigger the controller to display a response (such as changing an LED light color from green to red when the pressure is beyond a set threshold).

The housing is configured to connect to a needle cartridge. The needle cartridge may include at least one needle, a pressure inducer, and a de-bubbler. The pressure inducer may be a self-contained pump. However, it may alternately be a mechanical component or connection that connects to a subsystem within the housing to move media from the media cartridge to the needles. For example, the housing may include an actuator that connects to a series of gears and tubing that collectively form a peristaltic pump to move media through the system. The needle cartridge may be separable from the main body housing so that the needle cartridge can be disposed of after use and a new, sterile needle cartridge can be connected to the housing for use on a new person or with a new media. Alternately the needle cartridge may be sterilized and reused at later time.

The main body also accommodates a media cartridge that may be configured to removably connect to the needle cartridge and main body housing. The media cartridge holds the media to be injected through the needles of the needle cartridge. The media cartridge may include a media channel and a sight window to allow a user to see the media within the cartridge. It may further include a sensor that relays information about the volume of media within the cartridge to the controller which may then cause a display to provide information about the volume of media. The media cartridge may be configured to be refillable. Preferably, the media cartridge connects to the needle assembly though a snap-fit bayonet-style engagement. However, in certain embodiments, the media cartridge may attach in an alternate manner, such as by magnets, twist fit or sliding connection. This allows for the media cartridge to be connected and disconnected in a toolless manner and facilitates hot-swapping of media cartridges during a procedure.

The housing includes inputs, such as buttons or dials, that allow a user to control the operation of the system. By making selections through the inputs, the system transmits signals to the controller which in turn modifies the operation of the system. For example, the system may include a needle speed adjustor to control the speed or frequency of the needle penetrations. The system may also include a needle height adjustor (which may be manually operated) to adjust the penetration depth of the needles. The system may also include a media flowrate adjustor that controls the flow of media through the needles. For example, by varying the speed of a fluid pump, such as a peristaltic pump that draws fluid from the media cartridge and ejects media from the needles, the flow of fluid through the system can be manipulated.

The system may further be configured to display operational information, such as its operational speed. It may include a display indicating the speed or broadcast the operational information to a remote display, for example a Bluetooth® display connected to the system. The display may provide indications of other operational conditions such as the volume of fluid, the set flowrate, or current battery life.

In one embodiment the system may include a force sensor adapted to sense the amount of force being applied at the tip of the system when the dermal system is in use. In operation, the needle cartridge tip must be positioned against the skin so that when the needles protrude, they penetrate the skin. The user must apply pressure to prevent the needles from pushing the system away from the skin rather than properly penetrating the skin. However, too much force can overly stretch the skin or adversely affect the application of the media. The preferred operational range is no more than 1.5 pounds of pressure. The force sensor detects the proper amount of force to be applied and provides a visual or audio indication to the operator. For example, a three-tiered visual indication may be used, where the tiers are represented by different colors and indicate lack of force, proper force, and too much force. Alternatively, less or more colors could be included to correspond to different operational ranges. The indicator may be in the form of an LED array or annular ring that lights up with differing colors. The annular ring light allows the indicator to be seen regardless of orientation of the dermal system.

As discussed, the needle assembly is a needle cartridge that is preferably separable from the main body. Alternate types of needle assemblies may be alternately engaged with the main body to allow for differing types of procedures. For example, some procedures may utilize a relatively viscous media and require larger needles with large bores, while low viscosity media may be better adapted to application through small bore needles. The needle assembly's interface with the main body may include a sensor that indicates to the controller the type of needle cartridge attached (or that transmits one or more signals to the controller that the controller utilized to determine the type of needle cartridge attached). The needle sensor may reject needle cartridges that are not authorized to be used for the dermal system or prevent activation of the device if the needles are not properly installed. A similar interface may be provided for the media cartridge. This way the controller can be programmed to default to preset operational values (such as needle speed, depth, and pump speed) based on the type of needle assembly or media.

In certain embodiments, the main body of the system may be adapted to receive a sleeve to cover the main body. In one embodiment, the sleeve may be attached at the front end of the housing. In other embodiments the sleeve may be configured to be attached between the interface of the housing and the needle cartridge. For example, the sleeve may be placed over the main body, then needle cartridge and media cartridge may be fit overtop of the sleeve.

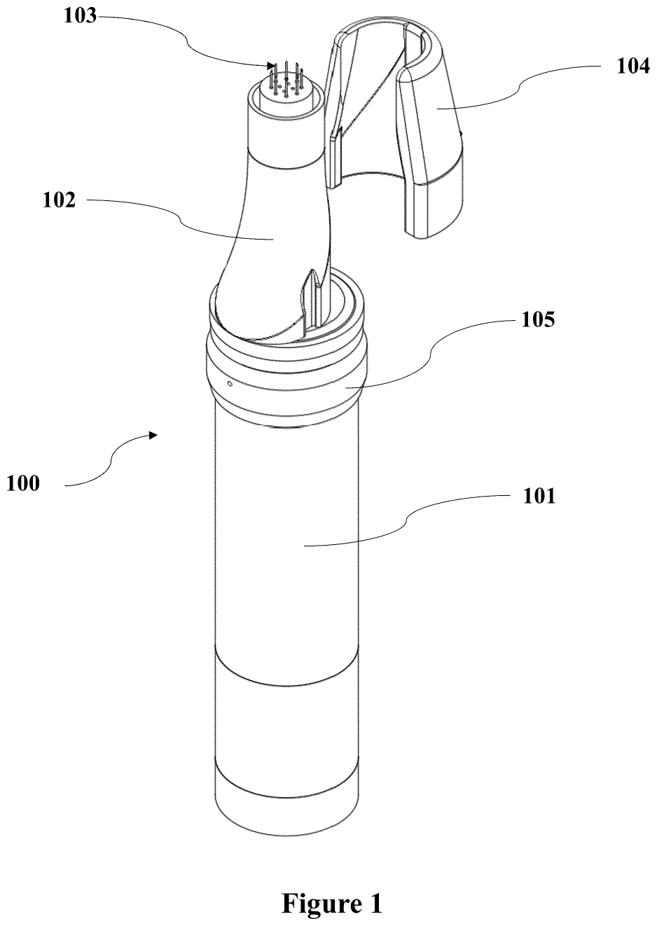

FIG. 1 is a general depiction of a dermal system 100. Main body 101 is formed of a housing that accommodates an actuator, controller, and power supply. Needle cartridge 102 is connected to one end of the main body. At the tip of the needle cartridge, a plurality of needles 103 are disposed in an array. A media cartridge 104 (shown disconnected) may be connected to the needle cartridge and main body. Collar 105 secures the needle cartridge to the main body.

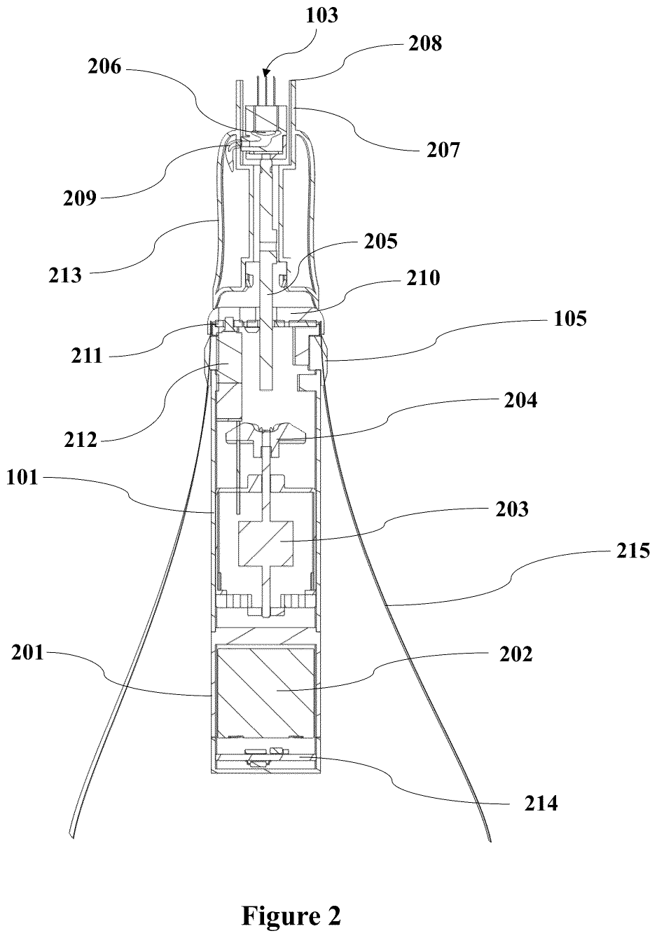

FIG. 2 is a cross-sectional view of the dermal system without the media cartridge. The base 201 of the main body 101 includes a cavity that houses a battery 202 and a control board 214. The main body also includes a cavity that houses actuator 203 (such as a motor). The motor connects to an interface 204 that engages with needle connection 205. In the embodiment of FIG. 2, needle connection 205 is a shaft that, in conjunction with the interface 204, translates motion of the actuator 203 into reciprocating linear motion that drives the needles to reciprocate.

Needle connection 205 is connected to needle manifold 206 which houses an array of needles 103. As the needle connection reciprocates, it reciprocates the needle array. The needle manifold and needle array are disposed within an open, hollow chamber defined by a manifold collar 207 having a rim 208. When the needles are fully retracted, the needle array seats within the hollow chamber, below the rim. This prevents damage to the needle tips. The base of the needle manifold is connected to a media tube 209 that is a fluid flow path that is supplied with media from the media cartridge. As the needle actuator reciprocates, the needles protrude from the hollow chamber, past the rim 208. By placing the rim against a person's skin and activating the dermal system, the needles are caused to protrude past the rim of the needle collar and penetrate into the skin where media may be injected.

As shown in FIG. 2, the needle cartridge connects to the main body via an interface plate 210 that is fitted with a force sensor 211. In use, an operator holds the dermal system by the main body. This allows the needle cartridge to “float”, separated from the grip of the operator. As the rim 208 of the manifold collar 207 is pressed against a person, that force is translated to the interface plate 210 and force sensor 211.

The main body houses a second actuator 212 that controls the flow of media through the system. For example, the actuator may be connected to a peristaltic pump assembly that flows fluid from the media cartridge through the media connection tube 209 and out the needles 103. Collar 105 provides secure engagement between the needle cartridge and main body. It may be a threaded collar that tightens around the main body. As the collar 105 is rotated, it adjusts the height of the needle cartridge with respect to the main body, which adjusts the position of the needle manifold. In this way the maximum protrusion of the needle array (i.e. the height of the needles, or alternately the maximum depth to which the needles may penetrate) may be adjusted. This allows precise adjustment of needle depth and therefore depth of media delivery, which is preferably no more than 3.0 millimeters. To protect the needle cartridge components, the needle cartridge is encapsulated in shroud 213. To treat the person's dermis, the needle cartridge rim 208 can be moved relative to the skin allowing for the needles to enter the skin at a determined maximum depth. In certain embodiments, the shroud 213 is adjusted relative to the needle assembly, therefore the needle assembly is stationary, and the shroud is used to gauge the height of the needles. Sleeve 215 may be utilized to cover the main body and collar 105 secures over the sleeve 215 and secures the needle cartridge to the main body.

In certain embodiments, the release of the media into the skin is timed with the insertion of the needles into the dermis. Once the needles retract from the dermis, the media pump is disengaged, and the surface tension of the media prevents the free flow of media out of the needle tips. The intermittent operation of the pump and penetration of the needles are synched by the controller. In other embodiments, the media continuously flows while the needles are repetitively cycled through penetrating and retracting.

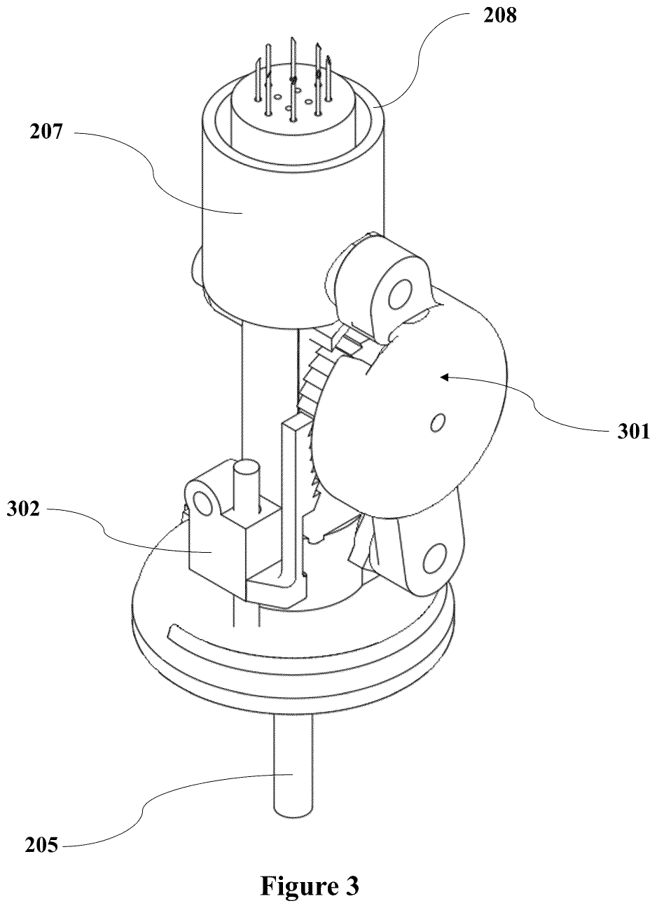

FIG. 3 is an alternate view of the needle cartridge 102 with the shroud omitted and showing the inclusion of a peristaltic pump 301. Tubing has been omitted for clarity. The pump 301 is encapsulated by shroud 213 and engages with actuator 212, for example a motor, through interface 302. The motor reciprocates interface 302 (such as a rachet mechanism) that rotates the pump 301 (such as a peristaltic pump) to pump fluid through the needles. In an alternate embodiment a transmission may be utilized instead of a second motor, and both the pressure inducer and the needle manifold are driven by a single actuator, such as actuator 203 although the transmission embodiment may be less adaptive to combinations of different needle arrays, media, and operational settings.

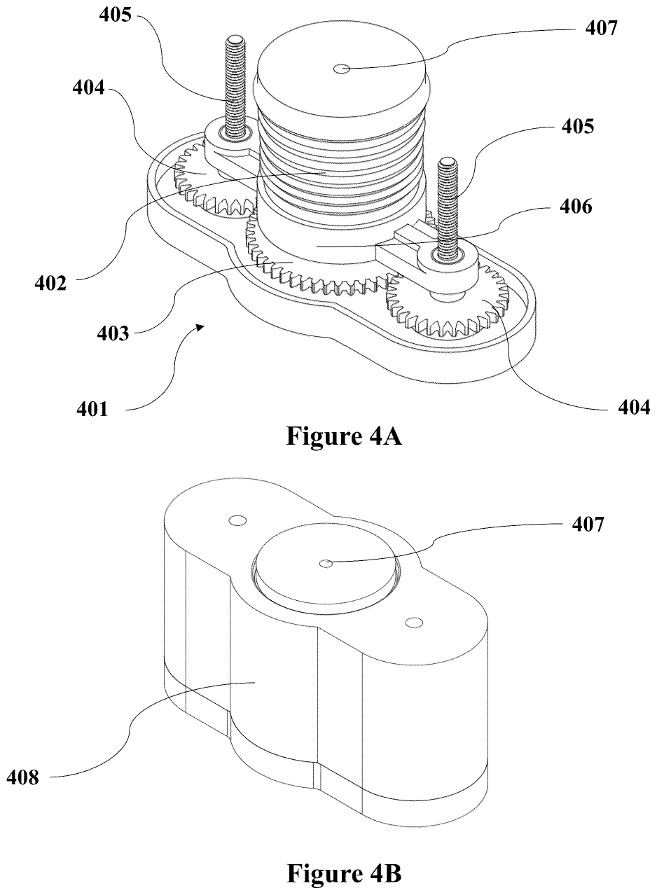

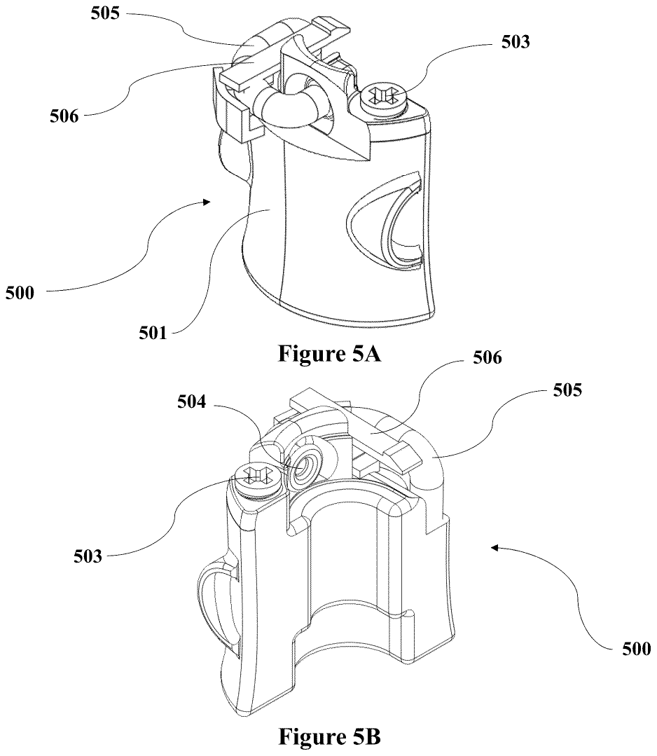

Alternate pumps to a peristaltic pump may be utilized. For example, in one alternate embodiment, flow is induced through a bellows system. As shown in FIG. 4A, a bellows pump 401 is shown. A bellows body 402 is configured to house media. A drive gear 403 is connected to the actuator 212 (not shown). As the actuator rotates the drive gear, the drive gear rotates carriage gears 404 and threaded rods 405. Carriage 406 is connected to the rods such that as the rods rotate in one direction, the carriage is moved up closing the bellows and ejecting a media through the exhaust port 407. In this embodiment, the bellows portion 402 houses the media. Thus, as the carriage is raised the media is expelled until the carriage reaches the top and the media is exhausted. The controller controls the operation of the motor to precisely rotate carriage gears 404. Intermittent rotation of the gears translates into precise doses of media which may be timed with the penetration of the needles. FIG. 4B is the bellows pump housing 408, which houses bellows pump 401. Utilizing the bellows pump is an alternative to having a separate media reservoir and pressure inducer (such as is shown in the combination of the FIG. 3 needle cartridge and FIGS. 5A and 5B media cartridge). It should be understood that other flow inducing mechanism could be utilized. However, by maintaining the operating motor of the flow inducing mechanism, such as the actuator 212, within the main body, separate from the needle cartridge and the media cartridge, the actuator is not discarded if the needle cartridge or media cartridge is disposed. This reduces the cost of replacement needle cartridges and media cartridges.

FIGS. 5A and 5B are depictions of a media cartridge 500. FIG. 5C is a cross sectional view of media cartridge 500. The cartridge includes a shroud or housing 501 that encapsulates a reservoir 502. FIG. 5D is a depiction of an example of the reservoir shown as a serpentine cavity or tube with two ends. The serpentine tube structure of the reservoir allows the reservoir to have a consistently small cross-sectional area along the length of the reservoir while still maintaining a significant total volume. The particular diameter of the cavity is adapted to the media housed within the reservoir such that application of a pressure source to push fluid, or suction force to draw fluid, prevents the mixing of air within the reservoir from mixing bubbles into the media. The reservoir houses media to be injected by the dermal system. The reservoir may have a vent 503 that selectively allows air to be drawn into the reservoir as media is expelled, and an exit port 504 through which media may be expelled from the reservoir and ultimately flowed out through the needles. Tubing 505 connects to the reservoir and the needle cartridge. A valve 506, such as a pinch valve, closes tube 505 when the cartridge is not engaged with the dermal system and opens when the cartridge is engaged. Alternate valving could be used to ensure that media is only permitted to flow when the cartridge is connected to the system. Detent 507 corresponds to a protrusion on the dermal system to ensure that the cartridge is properly aligned and may aid in snap-fit engagement of the media cartridge to the dermal system.

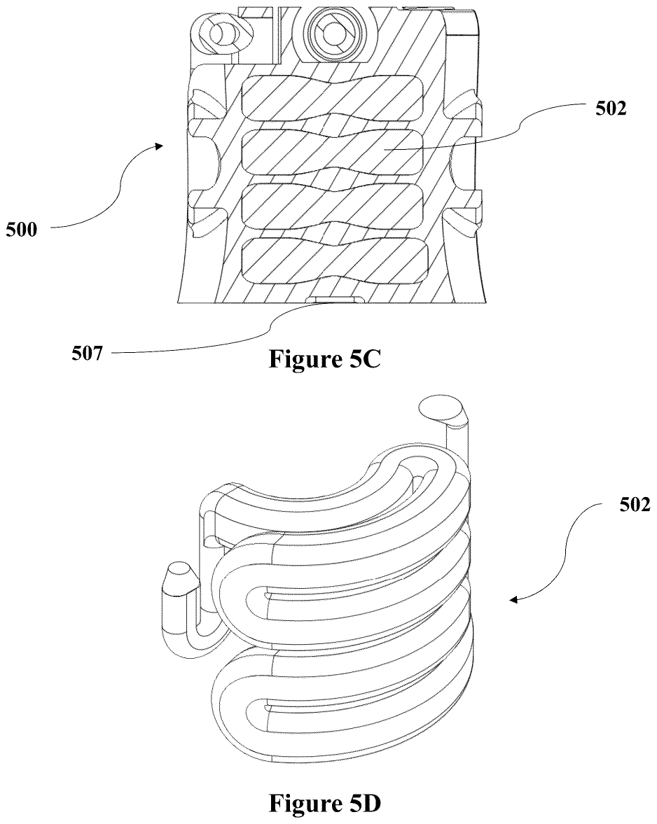

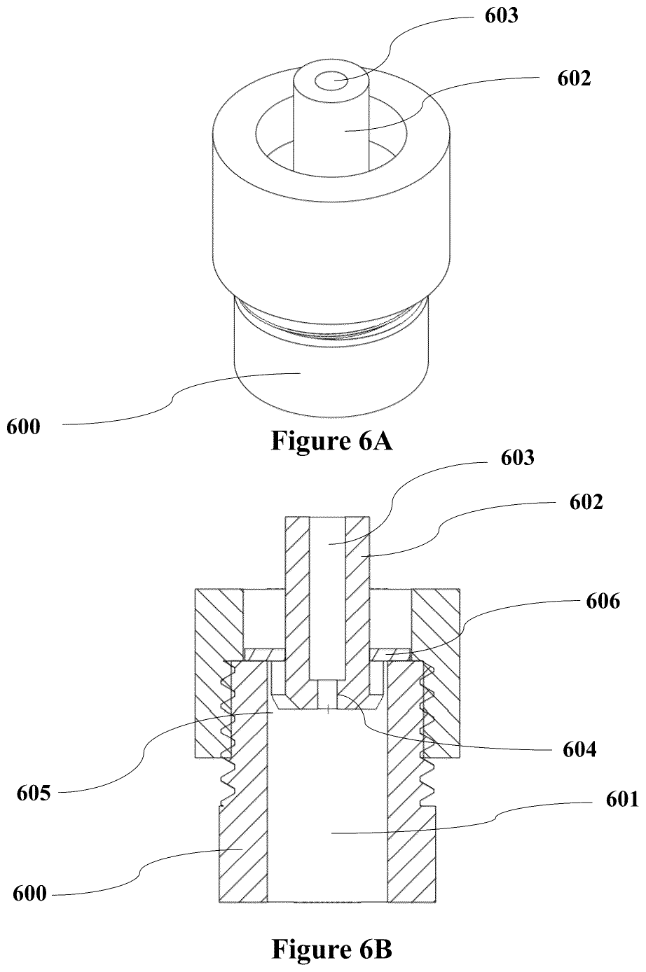

In some embodiments, the needle cartridge or media cartridge may further comprise at least one de-bubbling mechanism. Examples of embodiments of the de-bubbling mechanism are shown in FIGS. 6A, 6B, 7A, 7B and 7C. For example, in FIGS. 6A and 6B there is a de-bubbler that is formed of a large bore tube 600 that defines a media flow channel 601. Media from the media cartridge is flowed through tube 600 toward small bore tube 602. Tube 602 defines a media flow channel 603 that is smaller in cross-sectional area than the cross-sectional area of media flow channel 601. A restriction orifice 604 forms a passageway between tube 600 and tube 602 such that media can flow from tube 600 into tube 602. The external diameter of tube 602 is smaller than the internal diameter of tube 600 such that when tube 602 mates with tube 600, a gap 605 is formed between the tube 602 and a wall of channel 601. A gas-permeable membrane 606 seals around the exterior of tube 602 and across the gap 605. Thus, as media is forced through orifice 604 the restriction causes any gas bubble that may be within the media to migrate to the gap and through the gas permeable membrane to exhaust the gas while the media flows through the orifice 604. Media channel 603 connects to the needles so that the media flows through the needles without gas bubbles.

Alternately, as shown in FIGS. 7A, 7B and 7C, an upstream media tube 700 allows media to flow into a serpentine flow path 701. At least a portion of the serpentine flow path is formed of a gas-permeable membrane. In the embodiment of FIGS. 7A, 7B and 7C, the top and bottom of the serpentine flow path are each bound by a gas-permeable membrane, 706a, 706b (shown in FIG. 7A, but which have been omitted from the FIGS. 7B and 7C in order to show the serpentine flow path). Media flows from tube 700 thorough the serpentine path to restriction orifice 704. Media flows through restriction orifice 704 to downstream tube 702 and into media channel 703. The internal cross-sectional area of the serpentine path is preferably greater than the cross-sectional area of media channel 703 and both are greater than restriction orifice 704. Gas bubbles that may be in the media in the serpentine path pass through the gas permeable membranes that cover the serpentine flow path.

The gas permeable membrane may include silicone or polydimethylsiloxane (PDMS) membranes. In certain embodiments, a needle cartridge may include one or more de-bubblers to ensure uniform flow rates and expel gas prior to the media flowing through the needle cartridge. In one embodiment, a de-bubbler is positioned proximate to the needle manifold to allow for off-gassing of the media just prior to the media flowing through the manifold and through the needles. This helps ensure there is no unwanted amount of gas in the media and by extension no unwanted gas being injected through the needles and into the target skin area.

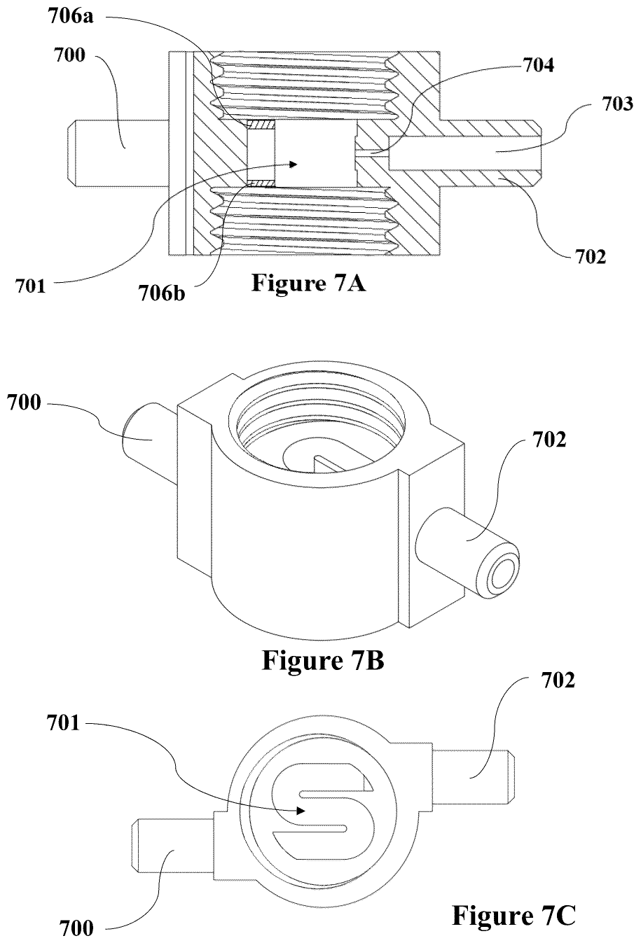

FIG. 8 is a simplified block diagram of the dermal system. The media cartridge, needle cartridge, and housing connect together to form the dermal system. The controller controls the pressure inducer, actuator, and the needle array actuator to flow media from the media cartridge to the needles and reciprocate the needles to apply treatments.

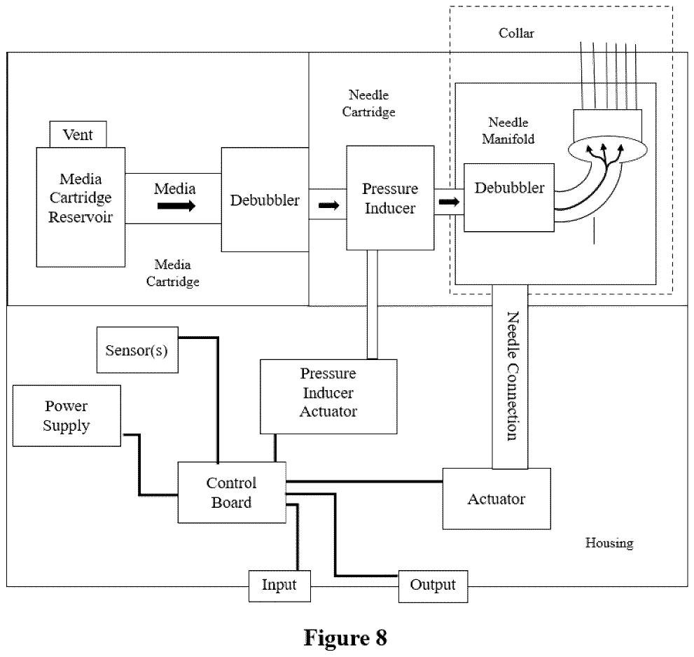

FIG. 9 is an enlarged view of the needle manifold assembly.

In one embodiment, the needle manifold 902 has multiple needle ports 901 which house needles 103 (though in an alternate embodiment the needle manifold may connect to a single needle). The needle ports 901 connect the needles to media manifold 902. During use, media flows through port 903 and the media manifold 902 evenly dispersed media into the needles 103 as the needle manifold 902 reciprocates. In some embodiments, the needle cartridge may comprise a single needle. Other embodiments may include a plurality of needles. In certain embodiments the needles may be solid. In other embodiments, as discussed above, the needles are hollow. In certain embodiments the needles may be textured. Textured needles may be used to hold and deposit additional media into the skin.

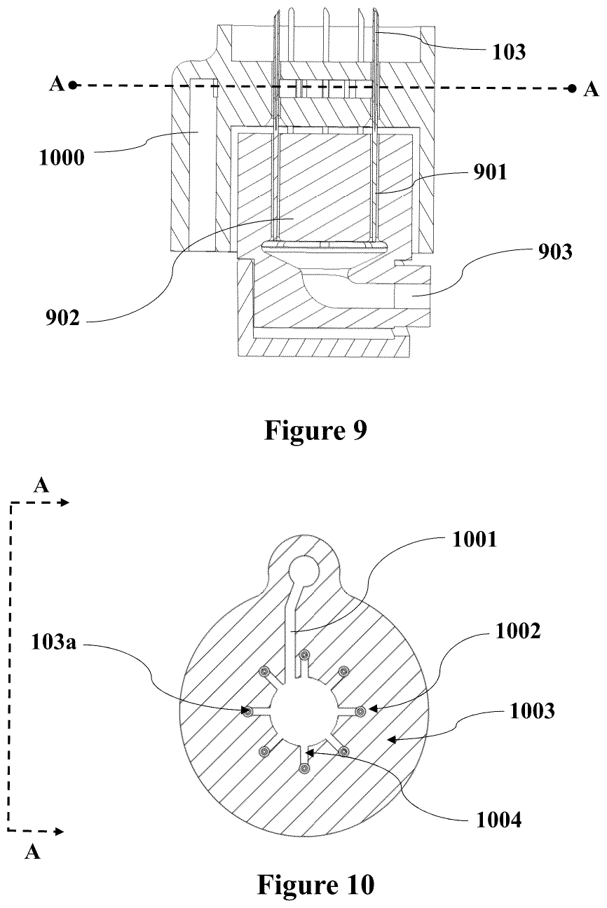

FIG. 10 shows an alternate fluid application system that may be utilized together or independently of the fluid application system of FIG. 9. It includes needle fluid dispersion path 1001 with a plurality of orifices 1002 through which needles may pass. The dispersion path is fed by flow path 1000 that channels fluid into the dispersion path 1001. Media flows from the media cartridge (not shown) through the dispersion path 1001 of a media well 1003. Media well 1003 may be in the form of a disk (though it need not be a traditional round disk) having orifices corresponding to the needles 103a. Each orifice (e.g. 1002) is connected to channel 1004 such that media may be flowed into each orifice. The orifices are sized such that a needle is able to reciprocate within the orifice without touching the sidewall of the orifice. The orifices are further sized such that when a needle is within the orifice, surface tension of the media between the media, sidewall of the orifice, and needle prevents the media from dripping due to gravity when the dermal device is inverted (i.e. when the needles are pointed toward the ground). As the manifold reciprocates, the shaft of a needle is caused to pass through an orifice and collect along its length a portion of media (and media is flowed to orifice), coating the needle which is then penetrated into the skin to deliver the media. This allows media to continually be supplied without the need to dip the needles or have excessive media pool at the treatment site.

In certain embodiments the media cartridge reservoir comprises serpentine media channel. The serpentine media channel's diameter may be between 1 mm and 25 mm. The diameter of the media channel will be determined by the viscosity of the media. By maintaining the media channel in proper proportions to the viscosity of the media, the media cartridge ensures that media flows out of the reservoir consistently and prevents air pockets forming within the media channel. Essentially, the media moves through the media channel like a core of media with air being allowed in through the vent cap to prevent a suction plug but being prevented from mixing with the media due to the restriction in the cross-sectional area of the serpentine media channel. In one embodiment the media cartridge may contain a sight-glass. The housing of the media cartridge may also be made out of a transparent material to show the amount of media in the media channel.

The unique structures of the dermal device allow for a method of use of the dermal system by selecting a needle cartridge corresponding to a desired dermal therapy, selecting a media cartridge containing media corresponding to the desired dermal therapy, connecting the media cartridge and needle cartridge to the housing of the dermal system, transmitting one or more signals from the media cartridge or needle cartridge to the controller, automatically setting the operating parameters of the needle actuator and pressure inducer to preset values based on the one or more signals, inputting alternative or additional operational parameters using inputs on the dermal system, transmitting one or more signals from the controller corresponding to the operation of the dermal system or an operational parameter of the dermal system to a display, activating the dermal system to flow media and reciprocate a needle of the needle cartridge, applying therapy to a desired location (i.e. piercing a person's skin with the needle(s) of the needle cartridge to deposit media into the dermis), stopping operation of the dermal system, removing the media cartridge or needle cartridge, replacing the media cartridge or needle cartridge, and repeating the process starting with the transmission of signals to the controller or from activating the dermal system to flow media and reciprocate a needle of the needle cartridge.

A person of ordinary skill in the art will recognize the interchangeability of various features from different embodiments disclosed herein. Similarly, the various features and steps discussed above, as well as other known equivalents for each such feature or step, can be mixed and matched perform methods and structures in accordance with principles described herein. Additionally, the methods which are described and illustrated herein are not limited to the exact sequence of acts described, nor are they necessarily limited to the practice of all of the acts set forth. Other sequences of events or acts, or less than all of the events, or simultaneous occurrence of the events, may be utilized in practicing the system.

Although the system has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, it is not intended that the invention be limited, except as by the appended claims.

What is claimed is:

1. A dermal media application system comprising:
a housing containing a motorized needle actuator and a controller;
a needle cartridge having a needle manifold that comprises a media well having a fluid dispersion path and a plurality of channels connected to the fluid dispersion path, and a plurality of needles connected to the needle manifold, wherein the plurality of channels comprises multiple single channels that each individually correspond to a single needle to provide fluid communication between the corresponding needle and the fluid dispersion path;
a media cartridge having a reservoir that accommodates a media;
a pressure inducer adapted to force media from the reservoir of the media cartridge to the needle cartridge;
wherein the needle cartridge and media cartridge are separate from the housing and adapted to connect to the housing and form a media flow path between the reservoir and the needle manifold; and
wherein, the controller is adapted to actuate the needle actuator to reciprocate the needle manifold and activate the pressure inducer to repeatedly force media to flow from the reservoir to the needle manifold during reciprocation.

2. The dermal media application system of claim 1 wherein the plurality of needles are solid, and wherein the controller activates the pressure inducer to flow media through the fluid dispersion path and into the channels while a portion of each needle is within its corresponding channel.

3. The dermal media application system of claim 1 further comprising a de-bubbler positioned within the media flow path and between the reservoir and the needle manifold.

4. The dermal media application system of claim 1 wherein the reservoir comprises a serpentine cavity having a vent at one end and an exit port at another end such that the exit port is adapted to connect to the media flow path.

5. The dermal media application system of claim 4 wherein a particular diameter of the serpentine cavity is determined at least in part based on the media housed within the reservoir such that application of a pressure to the reservoir by the pressure inducer sufficient to flow fluid within the reservoir prevents the mixing of air within the reservoir from mixing bubbles into the media.

6. The dermal media application system of claim 1 further comprising a force sensor, wherein the force sensor is coupled to the needle cartridge such that application of a compression force at a rim of the needle cartridge is sensed by the force sensor which is adapted to relay a signal to the controller.

7. The dermal media application system of claim 1 wherein the needle cartridge includes a manifold collar defining a hollow chamber; the manifold collar including a rim, wherein the actuator is adapted to reciprocate the needle manifold within the collar such that the entire of each needle retracts entirely below the rim and alternately a portion of each needle protrudes beyond the rim during the reciprocation. (Original) The dermal media application system of claim 7 wherein each channel includes an orifice at a distal end of the channel.

9. The dermal media application system of claim 7 wherein the needle extends beyond the rim no more than 3 millimeters. (Previously Amended) The dermal media application system of claim 6 further including an output display; wherein the controller is adapted to transmit a signal to the output display causing the output display to display an indication of the force sensed by the force sensor.

11. The dermal media application system of claim 1 wherein the pressure inducer is a pump, wherein a motor of the pump is positioned within the housing and the motor connects to a pumping mechanism within the needle cartridge or media cartridge.

12. The dermal media application system of claim 11 wherein the pumping mechanism is a peristaltic mechanism adapted to connect to the motor.

13. The dermal media application system of claim 11 wherein the pumping mechanism comprises a bellows forming a cavity having an exhaust port, drive gear, carriage, and a plurality of threaded rods, and wherein the motor is adapted to rotate the drive gear to move the carriage and compress the bellows such that a media within the cavity is expelled from the exhaust port.

14. The dermal media application system of claim 1 comprising a plurality of media cartridges, wherein a first media cartridge is adapted to connect to the needle cartridge and housing in a toolless manner, the first media cartridge is adapted to disconnect from the needle cartridge and housing in a toolless manner, and after the first media cartridge is disconnected, a second media cartridge is adapted to connect to the needle cartridge and housing in a toolless manner.

15. The dermal media application system of claim 8 wherein the orifices are sized such that each needle reciprocates within its corresponding orifice such that no portion of any needle touches a sidewall of its corresponding orifice.

16. The dermal medial application system of claim 15 wherein each orifice is sized such that when the needle is within the orifice, a surface tension of the media between the media, the sidewall of the orifice, and the needle prevents the media from dripping due to gravity when the dermal device is inverted.

17. (canceled)

18. (canceled)

19. (canceled)
